(19) United States
(12) Patent Application Publication
(10) Pub. No.: US 2017/0266349 A1
(43) Pub. Date: Sep. 21, 2017

(54) Transdifferentiated tissue graft

(71) Applicants: Claudia Eder, Vienna (AT); Michael Ogon, Vienna (AT)

(72) Inventors: Claudia Eder, Vienna (AT); Michael Ogon, Vienna (AT)

(21) Appl. No.: 15/500,097

(22) PCT Filed: Jul. 30, 2015
(86) PCT No.: PCT/EP2015/067546

§ 371 Date: Jan. 29, 2017

(30) Foreign Application Priority Data
Jul. 30, 2014 (EP) 14179132.7

(51) Int. Cl. A61L 27/38; C12N 5/077; A61L 27/36

(52) U.S. Cl. CPC A61L 27/3895; A61L 27/3645; A61L 27/3843; A61L 27/3886; C12N 5/0655; C12N 5/0654; A61L 2430/06; A61L 2430/38; C12N 2501/15; C12N 2506/13

(57) ABSTRACT

The invention provides a method of producing a connective tissue graft suitable for correcting a connective tissue defect, comprising determining the size and shape of a tissue defect, obtaining a fat tissue from a patient modelled to fit the size and shape of the tissue defect, contacting the fat tissue with one or more connective tissue specific growth or differentiation factors; and kits for such a method.

The present invention relates to bio-engineered tissue grafts, such as bone, cartilage, tendon, nerve or muscle grafts.

Bone grafts are applied to promote bone healing in spinal fusion, augment bone in maxillofacial surgery or in case of non-union of traumatic bone defects. Cancellous bone taken from the iliac crest is still considered as standard for spinal fusion, but is associated with high complication rates¹ Allograft bone is a frequently used alternative, but may lack osteogenicity and increase the risk of surgical site infection². Synthetic β-tricalcium phosphate bone grafts can be used either stand-alone or in combination with autologous stem cells, but implant failures have been reportet³. Tissue Engineered bone, fabricated by a combination of autologous cells and natural or synthetic bio-materials, might pose a potential alternative. However, these grafts have not proceeded to clinical practice so far, as the problem of sufficient vascularisation at the defect site is still unsolved⁴.

Another major clinical problem is the treatment of osteoporotic or traumatic vertebral fractures: Currently, recent fractures are treated by instillation of bone cement during a percutaneous Kypho- or Vertebroplasty procedure⁵. Complications of these procedures include cement extravasation, adjacent vertebra fracture and infection⁶. Another problem in using bone cements is the limited biocompatibility: In vitro studies on different PMMA cements demonstrated their potential to provoke cell damage and inflammation⁷. PMMA bone cements have no potential for shape modification after polymerization, making them unsuitable for treating children and young people in the period of growth. Resorbable calcium-phosphate cements can be used in this patient group, but show a leakage rate of 45% with unclear long-term clinical consequences⁸. Because of its low resistance against flexural, attractive and shear forces, there is a higher risk of cement failure and subsequent loss of correction⁹. A suitable biological therapy combining excellent biocompatibility with suitable mechanical stability does not exist so far.

Due to the poor intrinsic healing capacity, full thickness defects of articular cartilage remain an unsolved clinical problem. Surgical treatment options include bone marrow stimulation techniques such as drilling¹⁰ or microfracture¹¹. Both techniques establish a communication between the cartilage lesion and the bone marrow, allowing mesenchymal stem cells from the underlying bone marrow cavity to migrate into the defect¹². The transplantation of osteochondral cylinders from non-weight bearing areas of the joint¹³, represents another treatment option. None of these operative procedures leads towards restitutio ad integrum, as hyalinous cartilage is not obtained and the fibrocartilaginous repair tissue is incapable of withstanding mechanical stress over time¹⁴.

The transplantation of autologous chondrocytes (ACI), which have been isolated and expanded in vitro represents a biological approach towards cartilage repair¹⁵. ACI comprises a series of procedures: Cartilage tissue is harvested arthroscopically from a non-weight bearing area of the affected joint. The cartilage biopsy is dissected into small pieces and enzymatically digested to isolate articular chondrocytes, which are then expanded in vitro for several weeks. In a second surgical procedure, the defect is carefully debrided and covered by a periostal flap or biological membrane, beneath which the chondrocytes are injected. In a modification of that treatment, the chondrocytes are seeded onto a collagen matrix, which is then implanted¹⁶.

Both approaches carry difficulties: Periostal hypertrophy requiring revision surgery occurs in up to 15.4% of the cases, if a periostal flap is used to seal the defect¹⁵. Transient graft hypertrophy is also observed in 25% of the patients undergoing matrix assisted chondrocyte transplantation¹⁶. The usage of biological membranes, which are usually of bovine or porcine origin, may lead to allergic reactions and are therefore contra-indicated in patients with a known hypersensitivity to materials of animal origin¹⁷. As the products are not routinely screened for transmissible infectious diseases, they may pose a health risk to the health care provider¹⁸ and recipient.

In WO 2005/018549 A2 and in 2009 Evans et al.²³ describe the generation of activated muscle or fat grafts using a recombinant adenoviral vector for expressing BMP-2. This method bears the risk of viral infection²³, has a high risk profile and may lead to transplant rejection. Furthermore, adenovirus activated fat healed less quickly with high variability in healing bone consistency²³. Furthermore, adenovirus activated fat showed promise for use in tissue repair, but healed less quickly compared to muscle tissue. These differences might be method-specific by an eventual lower susceptibility of fat tissue to adenovirus infection. Orlicky and Schaak report the pre-adipocyte cell line 3T3-L1 to be inefficiently transfected by adenoviral vectors²⁵. In a review²⁴, ex vivo approaches were regarded to be cumbersome, very expensive and less attractive compared to in vivo cell activation methods.

WO 03/015803 A1 relates to providing mesenchymal cells to treat osteoarthrosis and articular defects in joints and further to produce transplants. The problem of providing suitable transplants is not addressed in this document.

Wang et al.²⁶ describes differentiating adipose-derived stem cells that were isolated from a rabbit and seeded into an acellular cartilage matrix.

Sandor et al.²⁷ and WO 2006/009452 A2 describe an artificial construct derived from isolated autogenous adipose stem cells. The cells were isolated and expanded ex vivo and seeded into a granular beta-tricalcium phosphate scaffold.

Salibian et al.²⁸ relates to stem cells in plastic surgery. Inok Kim et al.²⁹ and Jung et al.³⁰ relate to MSCs in fibrin glue.

Eun Hee et al.³¹ review uses of isolated adipose-derived stem cells.

Stromps et al.³² describes chondrogenic differentiation of isolated adipose-derived stem cells

Sujeong et al.³³ relates to neural differentiation of adipose tissue-derived stem cells. The cells were isolated from earlobes and cultured.

The use of cell cultures based on isolated adipose derived stem cells for bone cell formation was described by Halvorsen et al.¹⁹. Cells are removed from tissue by collagenase and cultured in vitro. However expansion of these cells in vitro is not efficient and the described projections of using such cells in a paste for bone repair failed.

Therefore there is a need to provide transplants that are well-tolerated by patients and provide an adequate tissue replacement fulfilling the requirements for strength and durability required by the repaired tissue as well as sufficient angiogenetic properties in case of vascularised recipient tissues.

In search for fulfilling these needs, the present invention provides a method of producing a bioengineered connective tissue graft by direct transdifferentiation of a donor connective tissue, preferably fat tissue, into another connective tissue type comprising culturing the donor connective tissue in vitro a) for at least 1 hour (preferably at least 2 days) or in vivo and/or b) by one or more connective tissue specific growth or differentiation factor. Further provided is a method of producing a connective tissue graft suitable for correcting a connective tissue defect, comprising determining the size and shape of a tissue defect, treating a donor connective tissue, preferably fat tissue, obtained from a patient by, in any order: modelling donor tissue to fit the size and shape of the tissue defect and contacting the fat tissue with one or more connective tissue specific growth or differentiation factors, thereby initiating differentiation of the tissue graft into another connective tissue. The inventive method uses whole tissues without isolating and culturing stem cells from the tissue. The inventive tissues sup-plied to the transdifferentiation step contains the cells in their original extracellular matrix and cellular organization, which is referred herein also as whole (donor) tissue. Consequently, the inventive method is referred to as “direct” transdifferentiation, i.e. “tissue to tissue”, in contrast to indirect differentiation via isolated cells. Isolated cells and grown products therefrom, be it in media or in an artificial scaffold, are not regarded as tissues according to the invention. According to the invention a connective tissue, the donor tissue (from a donor patient), is converted into another connective tissue, the graft tissue, which is different from the donor tissue type. The donor tissue is preferably fat. The inventive graft can be used to treat a connective tissue defect in a subject, e.g. by inserting the graft into the defect or applying the graft onto the defect. In particular, the invention provides in any method embodiment described herein a method of transdifferentiating fat tissue (as donor tissue) into another, non-fat tissue (the graft tissue). Also provided is a connective tissue specific growth or differentiation factor for use in a method as well as the connective tissue specific growth or differentiation factors for the manufacture of a composition for the use in such a method, such as a therapeutic method.

Also provided is an ex vivo or in vivo method for preparing the inventive graft, that is suitable to be used in this tissue defect correction therapy as well as a kit suitable for preparing and differentiating a donor, preferably fat, tissue into a suitable graft. Further aspects and preferred embodiments of the invention are described in the claims. All of these methods and embodiments are interrelated and may be combined with each other, such as the kit may be used in the inventive methods and vice-versa, the kit can be adapted to be suitable for performing any one of the inventive methods; the ex vivo method may be used as part of the therapeutic method; a graft produced in an ex vivo step can be used therapeutically or provided as a composition for such a therapeutic use. All preferred embodiments, described for any particular aspect shall also be regarded to be descriptive of any other inventive aspect, as is clear to one of skill in the art who will envisage immediately the generality of such embodiments. Further, each preferred embodiment can also be combined with each other preferred embodiment; in particular preferred is of course following all preferred recommendations described herein, except where explicitly exclusive.

The invention provides a method of producing a connective tissue graft suitable for correcting a connective tissue defect, which can be applied with or without a scaffold, obtaining a donor, preferably fat, tissue from a patient modelled to fit the size and shape of a tissue defect in a patient, contacting the donor, preferably fat, tissue with one or more connective tissue specific growth or differentiation factors (herein “incubation”), thereby initiating differentiation of the tissue graft. “Contacting” is a treatment of the tissue (or the cells within said tissue) with the respective growth or differentiation factors, whereby said tissue adapts to the new conditions caused by these factors, in turn leading to transdifferentiation.

Prior ex vivo method focused on either cell cultures of isolated cells or viral transfection. In vitro cell isolation, expansion in monolayer and re-differentiation prior to implantation is however cumbersome and can lead to dedifferentiation in monolayer cultures. The inventive transdifferentiation of whole tissue instead of isolated cells reduces these disadvantages and only requires minimal handling in vitro or ex vivo, which can be performed in a GMP-compliant, fully automated tissue processing device. E.g. in preferred embodiments cell culture medium need to be replaces or renewed at usual intervals such as 1-4 times a week.

Any type of fat tissue may be utilized a donor tissue, including, but not limited to subcutaneous depots from such areas as the chest, abdomen and buttocks, hips and waist; or ectopic or visceral fat. Although fat is a preferred donor tissue type in all embodiments of the invention, other connective tissues may be used as well as donor, e.g. cartilage, muscle, tendon, ligament or nerve donor tissues, for all embodiments of the invention instead of fat.

The tissue may be extracted from a patient using methods standard in the art for obtaining tissue for grafting. For example, such tissue may be surgically extracted using standard or minimally invasive surgical techniques. Minimal-invasive surgery may involve extracting the fat tissue through a natural or surgically created opening of small diameter in the body from a desired location of use so that surgical intervention is possible with substantially less stress being imposed on the patient, for example, without general anaesthesia.

In certain embodiments, tissue is removed from a patient in a size and shape suitable for implantation into a specific tissue defect. In other embodiments, the tissue is removed from the patient and then is altered to a desired size and shape ex vivo.

The donor, preferably fat, tissue may comprise stromal cells. It may also comprise adipocytes, such as white and/or brown adipocytes. Usually stem cells are present in the fat tissue that differentiates into cells specific for the connective tissue of interest, i.e. the tissue of the tissue defect. Such cells may be mesenchymal stem cells or stromal stem cells. Surprisingly also adipocytes, present in the inventive fat tissue, need not be removed but can remain and be embedded in the final differentiated tissue graft. The number of adipocytes may be at least 20%, at least 30% or at least 40%, at least 50%, at least 60%, at least 70% or more of all cells of the tissue graft.

Preferably, the adipocytes are stimulated to reduce or deplete their fat depots. This can be by a chemical or (cytokine or hormone) receptor stimulus or mechanical stimulation. A receptor stimulation includes contacting the donor tissue comprising the adipocytes with leptin. Mechanical stimulation includes kneading or dispersing the donor tissue comprising the adipocytes. The reduction or depletion of the fat depots is especially preferred in case of differentiating the tissue into a bone tissue, but also in case of cartilage tissue. The fat content can be reduced to a fat amount of less than 50% (w/w), preferably less than 30% or less than 20%, e.g. in the range of 50%-10%. Adipose tissue has a density of ˜0.9 g/ml. Fat reduction or depletion may result in a tissue with a density of at least 0.93 g/ml, preferably at least 0.95 g/ml, even more preferred at least 0.97 g/ml. The treatment can be to reach a density of e.g. 0.93 g/ml to 1.10 g/ml.

The order of the step of fitting the size and shape to a tissue defect and the step of contacting and thereby initiating differentiation of the donor tissue can be selected at will, e.g. a practitioner can first adjust the shape and then differentiate the cells or it is possible to first differentiate the cells and then shape the graft to fit the tissue defect. Of course, shaping can also be done before and after differentiation, e.g. providing a rough shape before and then fine tuning the shape after differentiation. Likewise, determining the size of the defect can be before or after differentiation. Preferred is before differentiation in order to select a donor tissue of adequate size, which may of course also be fine-tuned later to the size of the defect upon insertion to the defect.

The differentiation leads to a generation of increased number of cells of the graft connective tissue, including bone, cartilage, muscle (myogenic tissue), tendon (tenogenic tissue), ligament or nerve cells (neurogenic tissue), and preferably also extracellular matrix specific for the graft connective tissue. However, also the extracellular matrix of the donor tissue (especially fat tissue) may remain, at least in part, in the final graft tissue. By selecting suitable connective tissue specific growth or differentiation factors, that are known to a skilled person in the art, and/or incubation time suitable for said differentiation, the skilled person can steer the differentiation into a particular type of graft tissue. In particular, the graft connective tissue type may be bone or cartilage.

With “initiating differentiation of the tissue graft” it is meant that it is not necessary to fully differentiate all responsive cells of the donor connective tissue, preferably fat tissue, (e.g. stem cells) but it is usually sufficient to initiate the differentiation reaction so that the cell will continue differentiation even after (ex vivo) incubation, especially after insertion into the tissue defect. It is preferred that (ex vivo) differentiation occurs at least until the graft reaches a tensile strength of at least 5%, preferably at least 50%, of the tissue of the defect. In case of a bone graft, the graft tissue has a lower tensile strength than the bone to allow easy handling of the graft tissue, maintaining flexibility of the graft tissue. In case of bone preferred tensile strengths are about 5% to 15% of the tissue of the defect. Especially, without limitation, in case of cartilage, muscle, tendon, ligament or nerve graft targets, preferably the graft reaches a tensile strength of at least 25%, preferably at least 50%, of the tissue of the defect. Alternatively or in addition, it is preferred that differentiation occurs at least until the graft reaches a density of at least 20%, preferably at least 40%, e.g. 20% to 60%, of the tissue of the defect. Tensile strengths and densities as given herein refer to changes in the tensile strength or density as compared from the donor tissue of origin to the target graft tissue. The percentages define a change in the parameters (tensile strength or density) as a gradual change into the target tissue direction by said percentage value. The graft parameter can be calculated by A+(B−A)*P, with A being the parameter of the donor tissue, B of the target tissue of the defect and P the given percentage value. In case of bone grafts for bone defects, it is preferred that differentiation occurs at least until the graft reaches a mineralization contents of at least 20%, preferably at least 40%, e.g. 20% to 60%, of the tissue of the defect. Mineralization content can be determined by histology and determining the content of the mineralized area in a 2D slice.

For treatment of vertebral fractures, it is preferred that (ex vivo) differentiation occurs at least until the graft reaches a mineralization contents of at least 10%, preferably at least 20%, e.g. 10% to 30%, of the tissue of the defect. Generally, in case of fitting the graft tissue through a small channel, such as in case of vertebral fractures, it is preferred that the graft tissue is sufficiently elastic for transport through the channel in e.g. a folded state and the above mentioned lower mineralization is preferred.

The inventive method may further comprise placing the differentiated graft tissue into the tissue defect of a patient. Preferably the patient and/or the donor is a human or a non-human-animal. Non-human animals include mammals, such as include horses, cows, dogs, cats, pigs, sheep, birds, such as ostrich or parrot, reptiles, such as crocodiles. Preferably the patient with the tissue defect and the patient providing the donor tissue is the same patient (autologous tissue).

In one embodiment, the graft is incubated ex vivo for a period of time sufficient to allow at least a portion of the cells in the donor tissue to differentiate, or initiate differentiation, into a desired cell type for the tissue graft. Example time periods for the contacting step (incubation) is for 1 hour (h), to 10 weeks (w), preferably 1 hour to 6 weeks. Preferred time periods are at least 1 h, at least 2 h, at least 3 h, at least 5 h, at least 8 h, at least 12 h, at least 18 h, at least 24 h (1 day; 1 d), at least 30 h, at least 36 h, at least 2 d, at least 3 d, at least 4 d, at least 5 d, at least 6 d, at least 7 d, at least 8 d, at least 9 d, at least 10 d, at least 11 d, at least 12 d, at least 14 d. Alternatively or in combination with any of these minimum periods, the contacting step (incubation) is for at most 10 w, at most 8 w, 6 w, at most 5 w, at most 4 w, at most 3 w, at most 2 w, at most 10 d, at most 8 d, at most 6 d, at most 4 d, at most 3 d, at most 2 d, at most 1 d, at most 18 h. A preferred range is 2 d to 4 w.

In embodiments alternative to ex vivo differentiation or in combination therewith, the inventive graft is differentiated in vivo. Accordingly, a donor graft without ex vivo differentiation or a partially ex vivo differentiated graft is placed or implanted into a tissue defect and is stimulated to differentiate into the tissue type of the tissue of the defect. This can be done by administering the connective tissue specific growth or differentiation factor (specific for the tissue of the defect) to the implanted graft, e.g. by topical injections. Dosage and interval of the administration can be selected dependent on the tissue type and graft size. The connective tissue specific growth or differentiation factor can be the same as described further below for the ex vivo method, which is the preferred embodiment of the invention.

According to the invention, the cells to the donor tissue are preferably not isolated and expanded, but only transdifferentiated.

In preferred embodiments the connective tissue of the graft is cartilage (chondrogenic differentiated tissue). Differentiation may comprise the differentiation of cells of the fat tissue into chondrocytes and/or chondroblasts, preferably also their specific extracellular matrix. E.g. the graft tissue may comprise markers of cartilage extracellular matrix, as e.g. shown in FIG. 2a-c. The differentiation factor is then a chondrocyte differentiation factor. Such a factor or mixture of factors preferably includes TGF-beta. TGF-beta may include any one of TGFβ-1, TGFβ-2 or TGFβ-3 or a mixture thereof, such as of TGFβ-1 and TGFβ-2. Also, preferred is insulin-like growth factor (IGF). Further chondrogenic growth and differentiation factors include, BMP-2, BMP-4, BMP-6, BMP-7, BMP-9, dexamethasone, alpha-FGF, FGF-2, IGF-1, IGF-2, which may all be used optionally in addition (e.g. to TGF-beta) or as alternatives. In case of cartilage target graft tissue, it is preferred to culture the tissue in the absence of serum. Cartilage tissue quality can be further improved by addition of BMP-14 (GDF-5) during or after transdifferentiation.

In further preferred embodiments the connective tissue of the graft is bone (osteogenic differentiated tissue). Differentiation may comprise the differentiation of cells of the fat tissue into osteocytes and/or osteoblasts, preferably also their specific extracellular matrix, such as mineralization. The differentiation factor is then an osteogenic differentiation factor. Such a factor or mixture of factors preferably includes beta-glycerophosphate. Further osteogenic growth and differentiation factors include dexamethasone, bFGF, BMP-2, PGF, osteogenin, GDF-5, CTFG, which may all be used optionally in addition (e.g. to beta-glycerophosphate) or as alternatives. Especially preferred is serum as additive as described further in detail below. Particular preferred is a combination of beta-glycerophosphate, Dexamethasone and ascorbic acid, preferably further with serum.

In further preferred embodiments the graft connective tissue is tendon (tenogenic differentiated tissue). Differentiation may comprise the differentiation of cells of the donor tissue, preferably fat tissue, into tenocytes. The differentiation factor is then a tenogenic differentiation factor. Such a factor or mixture of factors preferable include mechanical in vitro stretching with or without any one or more of BMP-2, PGE₂, BMP-12, BMP-14, TGFβ₃ and platelet-rich plasma releasate, preferably also of serum. An example tenogenic differentiation medium contains DMEM-F12 supplemented with 1% FCS and BMP-12, preferably about 10 ng/ml BMP-12.

In further preferred embodiments the graft connective tissue is muscle (myogenic differentiated tissue). Differentiation may comprise the differentiation of cells of the donor tissue, preferably fat tissue, into myocytes. The differentiation factor is then a myogenic differentiation factor. Such a factor or mixture of factors preferable includes any one or more of 5-azacytidine, amphotericin B, bFGF and preferably also serum.

In further preferred embodiments the graft connective tissue is nerve (neurogenic differentiated tissue). Differentiation may comprise the differentiation of cells of the donor tissue, preferably fat tissue, into neural cells. The differentiation factor is then a neurogenic differentiation factor. Such a factor or mixture of factors preferable include any one or more of FGF-2, retinoic acid, 2-mercaptoethanol, hydrocortisone, cAMP, aFGF, Shh, brain derived neurotropic factor, nerve growth factor, vitronectin, AsA, 3-isobutyl-1-methylxanthine, forskolin and phorbol myristate acetate (preferably 20 nM thereof), and preferably also serum.

In further preferred embodiments the graft connective tissue is a ligament. Differentiation may comprise the differentiation of cells of the donor tissue, preferably fat tissue, into ligament cells. The differentiation factor is then a fibroblastic differentiation factor. Such a factor or mixture of factors preferable includes any one or more of TGF-β1, IGF-1, PDGF, BMP-12, bFGF and insulin, preferably also serum.

For cartilage differentiation, growth and differentiation factors may include one or more of the group selected from dexamethasone, ascorbate 2-phosphate, insulin, selenious acid, transferrin, sodium pyruvate and transforming growth factor β (TGF-β), BMP-14 and/or insulin-like growth factor (e.g. IGF-1). Especially preferred is TGF-β and/or IGF-1. Especially efficient differentiation can be achieved with all of these components.

Additional nutrients that may also be included include Dulbecco's modified Eagle's Medium and Ham's nutrient mix F12, any proteinogenic amino acid, e.g. L-glutamine.

For osteogenic (bone) differentiation, the cartilage growth and differentiation factors may be used with additional bone growth and differentiation factors, given that cartilage development is a precursor to bone development.

Osteogenic differentiation factors are e.g. described in Ref 21, and may comprise 1 to 1000 nM dexamethasone (Dex), 0.01 to 4 mM L-ascorbic acid-2-phosphate (AsAP) or 0.25 mM ascorbic acid, and 1 to 10 mM beta-glycerophosphate (beta GP). It may comprise DMEM base medium plus 100 nM Dex, 0.05 mM AsAP, and 10 mM beta GP.

Preferably the bone differentiation and growth factors include one or more of the group selected from ascorbic acid, any proteinogenic amino acid, e.g. L-glutamine, dexamethasone, β-glycerolphosphate and leptin. Especially preferred are beta-glycerolphosphate and/or leptin. Especially efficient differentiation can be achieved with all of these components.

Nutrients as in Dulbecco's Modified Eagle's Medium (DMEM) and/or serum are also preferred to be used during bone differentiation. DMEM provides basic nutrients, including amino acids, that can be used in any method of the invention for culturing any pre-differentiated fat tissue, during differentiation and afterwards.

Preferably a serum, especially autologous serum from the recipient of the transdifferentiated tissue graft, is added to the donor, preferably fat, tissue during the differentiation step, that is preferably performed in culture ex vivo. Serum can be a mammalian serum, such as bovine serum, especially preferred fetal calf serum or fetal bovine serum, but preferably human serum in case of human patients. Serum may be supplied in a concentration of between 1% to 80% (v/v), preferably between 2% to 60%, 3% to 50%, 4% to 40%, 5% to 30%, especially preferred 6% to 20%. Serum is usually used only to maintain certain cells viable or proliferative—even cartilage cells, which however dedifferentiate into other cells and/or reduce their collagen and glycosaminoglycane synthesis in the presence or serum. Serum is preferably not used for cartilage grafts. Transdifferentiation usually is independent of serum. Serum may be used during bone, muscle, tendon, ligament or nerve production according to the inventive method. Further steps may be used to advance differentiation into cells of these tissues.

Preferably IGF, especially IGF-1, is used for bone differentiation.

Preferably the connective tissue specific growth and/or differentiation factors are provided extrinsically to the tissue, e.g. the tissue is contacted with these factors and the factors are not recombinantly expressed in the cells of the donor or graft tissue.

Surprisingly, it was found that the bone differentiation could be facilitated without a Bone Morphogenetic Protein, such as BMP-2. Therefore, in preferred embodiments to the invention a BMP, or a nucleic acid encoding a BMP, is not added to the fat tissue for cartilage and/or bone differentiation. In other embodiments, a BMP, e.g. BMP-5 or BMP-7, may be used. BMPs are BMP1, BMP2, BMP3, BMP4, BMP5, BMP6, BMP7, BMP8a, BMP8b, BMP10, BMP15.

Preferably nucleic acids, e.g. transgenes, are not used as growth or differentiation factors. The inventive differentiation or growth factors are proteins or peptides. In addition or alternatively small organic molecules with a size of at most 10 kDa, preferably at most 5 kDa, especially preferred at most 2 kDa, can be used.

Preferably the connective tissue specific growth or differentiation factors comprise ascorbic acid or an ascorbic acid ester, preferably ascorbate-2-phosphate, or any pharmaceutically acceptable salt thereof. Ascorbic acid and esters thereof, such as L-ascorbic acid 2-phosphate, stimulates collagen accumulation, cell proliferation, and formation of a three-dimensional structures by skin fibroblasts. Thus ascorbic acid and its derivatives are preferred components as connective tissue specific growth or differentiation factor or as an additive in a mixture of such factors—in any of the inventive methods and any embodiment thereof, including the cartilage or bone differentiation.

Preferably the differentiation (incubation) is done at a temperature of between 30 to 40° C. Also preferred, the differentiation (incubation) is done at an atmosphere comprising 0.01% to 10% (w/v) CO₂. Also preferred, the differentiation (incubation) is done at an atmosphere comprising between 70% to 98% humidity. Preferably, but not necessarily, a combination of these parameters is used.

In the inventive method, cells of the donor and graft tissue remain viable, at least the cell that are transdifferentiated within the tissue.

In a particular aspect, the present invention provides an ex vivo method for preparing a donor, preferably fat, tissue into a differentiated graft suitable for connective tissue repair, comprising contacting the donor, preferably fat, tissue with one or more connective tissue specific growth or differentiation factors, thereby initiating differentiation of the tissue graft, wherein said contacting is for a time period of between 1 hour to 4 weeks at a temperature of between 30 to 40° C., 0.01% to 10% (w/v) CO₂ and between 70% to 98% humidity, preferably wherein the method is further defined by further steps as described above or in the following. Especially, the connective tissue specific growth or differentiation factors consist of proteins, peptides and small molecules with a size of at most 10 kDa, e.g. no recombinant expression in the cells of the fat tissue is used.

The donor or graft tissue can be dissected into slices of the desired size to facilitate minimally invasive insertion if desired. Desired size length may be 0.3 mm to 10 mm. Preferably, the inventive donor prior or during transdifferentiation or the graft tissue has a size of 0.001 mm³ to 1000 cm³, preferably of a size of 0.01 mm³ to 100 cm³, or a size of 0.1 mm³ to 10 cm³, a size of 1 mm³ to 1 cm³, or of 10 mm³ to 100 mm³.

The differentiated tissue graft is inserted into or onto the tissue defect, and further preferably wherein the insertion is fixed by a tissue sealant, preferably a fibrin glue.

Any one of the above described differentiation steps, i.e. the incubation are provided as a further aspect of the invention in an ex vivo or in vivo method for transforming a fat tissue into a tissue graft of a connective tissue, comprising said step of incubation.

Preferably, any one of the inventive method further comprises contacting the tissue with a tissue sealant, preferably after treatment with said connective tissue specific growth or differentiation factors and/or after an optional step of adjusting the size and shape of the tissue graft to fit a connective tissue defect of a patient. Adjusting the shape and size means that the tissue graft will fit into or onto the defect space to allow healing of the defect, i.e. attachment of the graft to the neighbouring connective tissue. Treatment with the tissue sealant allows firm attachment of the graft to the surrounding connective tissue and enhances growth of cells of the connective tissue. The tissue sealant can also be coated into the tissue defect. In any way the tissue sealant is used to connect the graft with the surrounding connective tissue.

Generally treatment of a tissue defect can include implanting the inventive graft in a volume of missing tissue (tissue defect) that is e.g. naturally, due to injury or due to surgery missing. The graft can also be used to treat a superficial tissue defect onto which the graft is fixated and from which a strengthening effect occurs to the underlying defect, such as by migration of activated or differentiated stem cells from the graft tissue into the defect.

Especially preferred, the graft tissue contains blood vessels. The blood vessels are maintained from the donor graft tissue and do not need to be regrown. Such blood vessel in the graft can connect with blood vessels in the vicinity of the tissue defect after transplantation and allow good bonding of the graft to the surrounding or adjacent tissue harbouring the original defect.

Defects can be in many tissues, which may require e.g. orthopaedic surgery, maxillofacial surgery, dentistry or plastic and reconstructive surgery.

Example defects and therapies with the inventive tissue graft include, sorted by graft or defect tissue:

A cartilage graft tissue can be used in the treatment of cartilage defects. Cartilage types of defects to be treated include focal cartilage lesions such as osteochondritis dissecans or traumatic cartilage injury, for example in the knee joint or talus; Osteoarthritis, especially cartilage abrasion due to osteoarthritis; Intervertebral disc regeneration; Meniscus regeneration; intervertebral disc lesions caused by nucleus pulposus prolapse and subsequent microdiscectomy. The transdifferentiated tissue graft can serve as a biologic nucleus pulposus substitute. It can be used in the treatment of degenerative intervertebral disc disease by implantation of a chondrogenic transdifferentiated graft as nucleus pulposus substitute. Cartilage graft tissues can further be used in the treatment of traumatic or degenerative meniscus tears. The transdifferentiated graft can be sutured into the meniscus defect after partial resection of the torn meniscus.

A ligament graft tissue can be used in the treatment of ligament defects, including treatment of traumatic cruciate ligament tears in the knee joint; treatment of traumatic tears of the lateral ankle ligaments.

A tendon graft tissue can be used in the treatment of tendon defects, including treatment of traumatic or degenerative rotator cuff tears; treatment of achilles tendon tears.

A bone graft tissue can be used in the treatment of bone defects including bone surgery, such as in spinal fusion in case of a spinal deformity or degenerative disc disease: The osteogenic transdifferentiated graft can be used for cage and intervertebral space filling after removal of the intervertebral disc and preparation of the intervertebral space. The graft can also be used either alone or in combination with BMP to achieve spinal fusion via graft apposition onto the spine. The graft can be used in the treatment of non-union after bone fracture or it can be grafted into a damaged area to facilitate bone healing. It can be used to treat osteoporotic defects or for bone augmentation, including prophylactic treatments, such as in Kypho- and Vertebroplasty either as a treatment of a vertebral body fracture or for prophylactic bone augmentation. The osteogenic transdifferentiated graft can be inserted into the vertebral body and contribute to biological fracture healing. It can be used for treating bone defects normally requiring bone grafting, such as aneurysmatic and juvenile bone cysts or for bone augmentation such as prior to insertion of dental implants. E.g. it can be used for sinus lifting in dentistry. The bone with a defect to be treated according to the invention can be a long bone, short bone, flat bone, sesamoid bone or irregular bone according to the common classification of bone types.

A graft prepared by the inventive method can be provided for use in any therapeutic tissue defect treatment.

Also provided in a further aspect is a kit suitable for performing a method of the invention, in particular a method of differentiating the cells of the fat tissue into the suitable connective tissue graft with differentiated cells and optionally further suitable for attaching the graft into a tissue defect. The kit may comprise a connective tissue specific growth or differentiation factor—preferably as described above—and a tissue sealant, preferably fibrin glue. Any other component as described above may be included. Further provided may be incubation containers, such as flasks or dishes. Also provided may be components to adjust a suitable atmosphere, such as a CO₂ flask.

The kit may further comprise a cartilage or bone tissue label or marker, which is suitable to monitor the progress of differentiation and to evaluate if a given differentiation stage of the fat tissue, being transformed into the connective tissue graft, is sufficient for insertion into a defect. Stages of differentiation may be as described above.

The present invention is further characterized by the following figures and examples, without being limited to these embodiments of the invention.

### FIGURES

FIG. 1: Smooth surface transformation of chondrogenic transdifferentiated fat graft (a) compared to corresponding control (b) showing irregular, uneven surface formation

FIG. 2: Alcian Blue Glycosaminoglycan staining of a chondrogenic transdifferentiated fat graft (a) and the corresponding control (d); Bismarckbrown staining of fat graft (b) and control (e); Safranin O staining of fat graft (c) and control (f).

FIG. 3: Glycosaminoglycan content of a chondrogenic transdifferentiated fat graft.

FIG. 4: Mineralisation of an osteogenic transdifferentiated fat graft indicated by positive von Kossa staining (a) and Alizarin red staining (b). The corresponding controls (d,e) show no signs of mineralization. Azan staining demonstrates an increase in collagenous tissue in the transdifferentiated fat graft (c) compared to the undifferentiated control (f).

FIG. 5: Quantification of mineralisation in 5 μm sections of an osteogenic transdifferentiated graft

FIG. 6: Evaluation of angiogenesis and tissue integration of an osteogenic transdifferentiated graft using the HET-CAM angiogenesis assay. The graft demonstrates good tissue integration and is connected to the vessel system of the recipient after 5 days in vivo (a,b). Numerous blood vessels are visible within the graft (c; arrows). Osteogenic differentiation is maintained as demonstrated by positive von Kossa staining (d).

FIG. 7: Hyalinous cartilage of the knee joint 14 days after implantation of a chondrogenic transdifferentiated fat tissue graft. The graft is well integrated into the recipient tissue.

FIG. 8: Isolated, neurogenic differentiated mesenchymal stem cells exhibit typical neuron and axon formation (FIG. 8a, arrows). Axon and neuron formation is not observed in the control group (FIG. 8b). The neurogenic transdiffereniated fat graft demonstrates positive Nissl body staining (FIG. 8c, arrows), which is not observed in the control group (8d).

FIG. 9: a) Control fat tissue demonstrating the typical adipose tissue phenotype. b) Neurogenic transdifferentiated fat pad after 6 weeks: The fat vacuoles have been replaced by neurogenic tissue indicating positive cresylviolett staining. c) The zonal differentiation of peripheral nervous tissue, containing perineurium (black arrow) and epineurium (grey arrows) as well as concomitant blood vessels (yellow arrows) can be observed. d) Nissl bodies within the perineurium are visible in the neurogenic transdifferentiated sample (arrows).

FIG. 10: a) Differentiation of isolated mesenchymal stem cells towards a tenocytic phenotype two weeks after initiation of differentiation. b) unaltered phenotype of the control group. c) Islands of tenocytic differentiated cells showing circular orientation were present in the transdifferentiated fat pads, but not in the control tissues (d).

FIG. 11: Myogenic differentiation: a) Early differentiation towards oriented myocytes; b) no differentiation in the control group. c) Fat vacuoles were partially replaced by muscle tissue demonstrating longitudinal orientation and positive Goldner staining; d) muscle tissue formation was absent in the control sample.

### EXAMPLES

### Example 1

### Transdifferentiation of Fat Tissue into a Hyaline Cartilage Graft

### Fat Graft Preparation:

A small fat biopsy obtained during spinal decompression surgery was placed in a sterile container for transportation to the tissue culture laboratory. The sample was washed in sterile saline solution to remove contaminating erythrocytes. After passing the contamination check, the sample was divided into two parts. Part A (Transdifferentiation sample) was incubated in a commercially available chondrogenic differentiation medium (Promocell, Heidelberg/Germany) intended to use for mesenchymal stem cell differentiation. To obtain mesenchymal stem cell differentiation, cells are usually placed in aggregate or pellet cultures in a defined medium containing dexamethasone, ascorbate-2-phosphate, insulin, selenious acid, transferrin, sodium pyruvate and transforming growth factor β (TGF-β)¹⁵. Part B (control) was incubated in a 1:1 mixture of Dulbecco's modified Eagle's Medium and Ham's nutrient mix F12 supplemented with 10% Fetal Calf Serum and 2 mM L-glutamine. To prevent bacterial contamination, 50 pg/ml Gentamycin was added to both culture media. Incubation took place at 37° C., 5% CO₂ and 90% humidity for 2-3 weeks. Medium was exchanged twice a week.

### Histological Evaluation:

At the end of the incubation period, samples were fixed in 4% formaldehyde, washed in phosphate buffered saline and drained in ethanol in ascending concentrations. Tissues were embedded in paraplast and 5 μm sections were prepared. Chondrogenic differentiation was evaluated via Alcian Blue, Bismarck Brown and Safranine O staining.

### Evaluation of Glycosaminoglycan Syntheses:

After 2 weeks of transdifferentiation, samples were digested over night in 1 mg/ml Proteinase K dissolved in 50 mM Tris containing 1 mM EDTA. Glycosaminoglcan content was measured using the Dimethyl-Methylenblue assay and absorbance was read at 525 nm. Shark chondroitin sulphate was used for generation of the standard curve.

### Morphological Results:

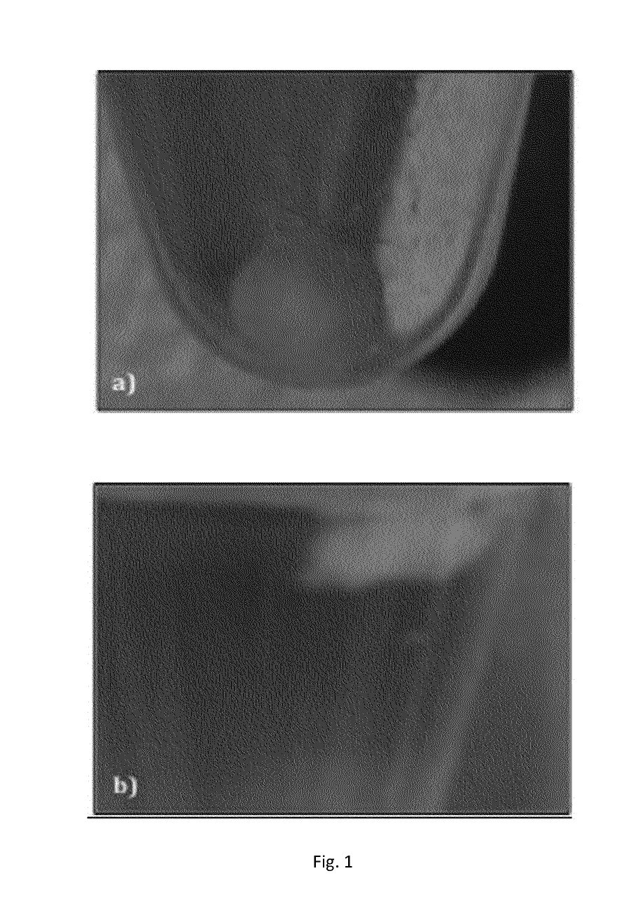

After 3 weeks of transdifferentiation in vitro, the fat tissue showed a compact, spherical morphology with smooth surface remodelling (FIG. 1).

### Histological Results:

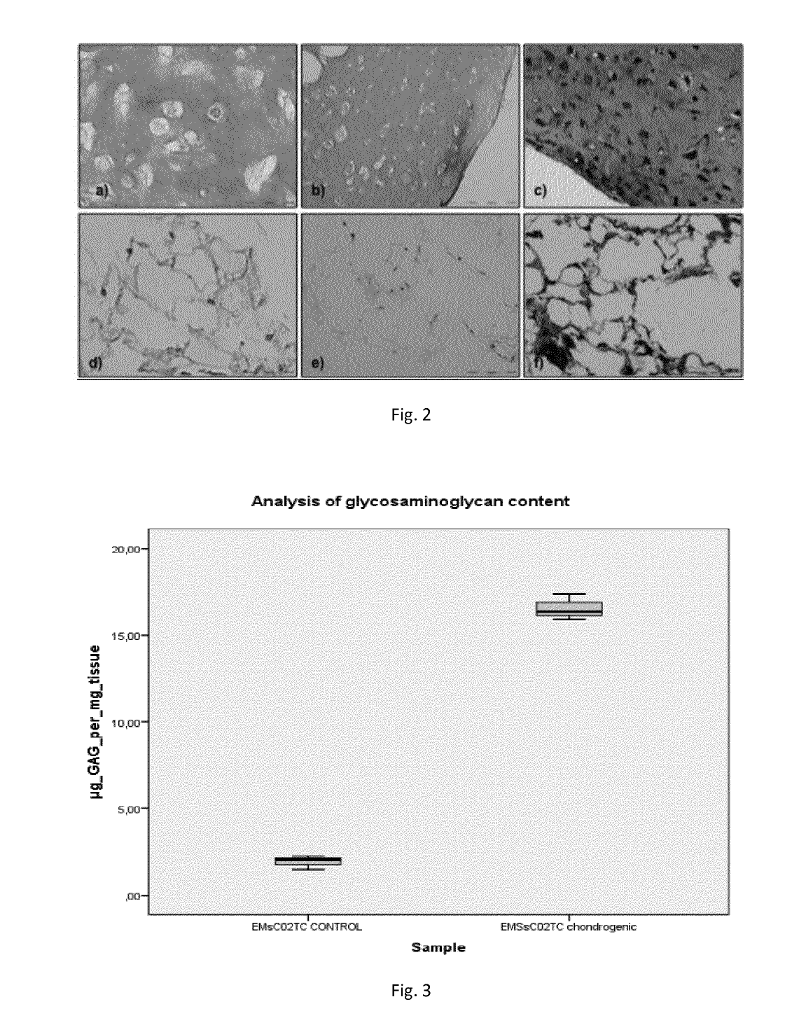

Histological staining for chondrogenic differentiation was positive in the transdifferentiated fat tissue: Glycosaminoglycan synthesis could be detected via Alcian Blue staining. Proteoglycans were further visualized via positive Safranine O staining, and Positive Bismarck-Brown staining indicated the presence of an extracellular matrix typical for cartilaginous tissue (FIG. 2).

### Evaluation of Glycosaminoglycan Syntheses:

Two weeks after inition of the transdifferentiation process, transdifferentiated fat grafts contained 16.56 μg glycosaminoglycans/mg tissue while the controls only showed an average glycosaminoglycan content of 1.92 μg/mg (p<0.0001; FIG. 3).

### Example 2

### Transdifferentiation of Fat Tissue into a Bone Graft

### Fat Graft Preparation:

A small fat biopsy obtained during spinal decompression surgery was placed in a sterile container for transportation to the tissue culture laboratory. The sample was washed in sterile saline solution to remove contaminating erythrocytes. After passing the contamination check, the sample was divided into two parts. Part A (Transdifferentiation sample) was subjected to repeated mechanical stimulation followed by incubation in osteogenic differentiation medium. Osteogenic differentiation medium consists of Dulbecco's Modified Eagle's Medium (DMEM) supplemented with 10% fetal calf serum, 0.05 mg/ml ascorbic acid, 2 mM L-glutamine, 1 μM dexamethasone, 10 mM Na-β-glycerolphosphate and 1 μg/ml leptin. Part B (control) was incubated in a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 95% humidity for 3 weeks. Medium was exchanged twice a week.

### Histological Evaluation:

At the end of the incubation period, samples were fixed in 4% formaldehyde, washed in phosphate buffered saline and drained in ethanol in ascending concentrations. Tissues were embedded in paraplast and 5 μm sections were prepared. Samples were stained with Azan, von Kossa and Alizarin Red.

### Histological Results:

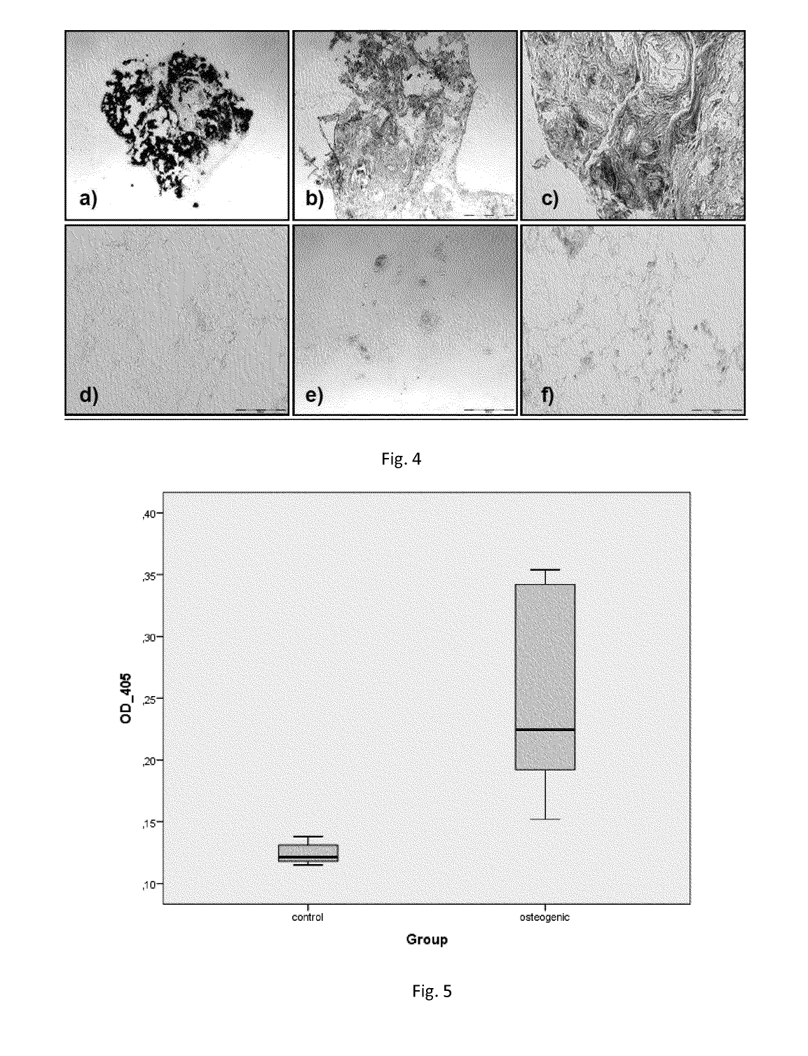

The transdifferentiated fat graft shows an increase in collagen content and signs of mineralization as indicated by positive von Kossa and Alizarin red staining (FIG. 4).

### Quantification of Mineralization:

The degree of mineralization was quantified from 5 μm sections by determining the optical density (OD) of alizarin red staining after 3 weeks of osteogenic transdifferention.

### Alizarin Red Staining Results:

Average OD of the osteogenic transdifferentiated graft was 0.25 per 5 μm section. OD of the corresponding control section was 0.12 (p<0.005; FIG. 5) Evaluation of angiogenesis and tissue integration:

Angiogenesis and tissue integration were evaluated using the HET-CAM (Hen Egg Test—Chorionallantoic Membrane) assay. The osteogenic transdifferentiated grafts were heterotopically implanted onto the exposed chorionallantoic membrane of fertilized, specific pathogen free chicken eggs. 5 days after implantation, the graft bearing area of the CAM was excised and processed for histological analysis.

### HET-CAM Testing Results:

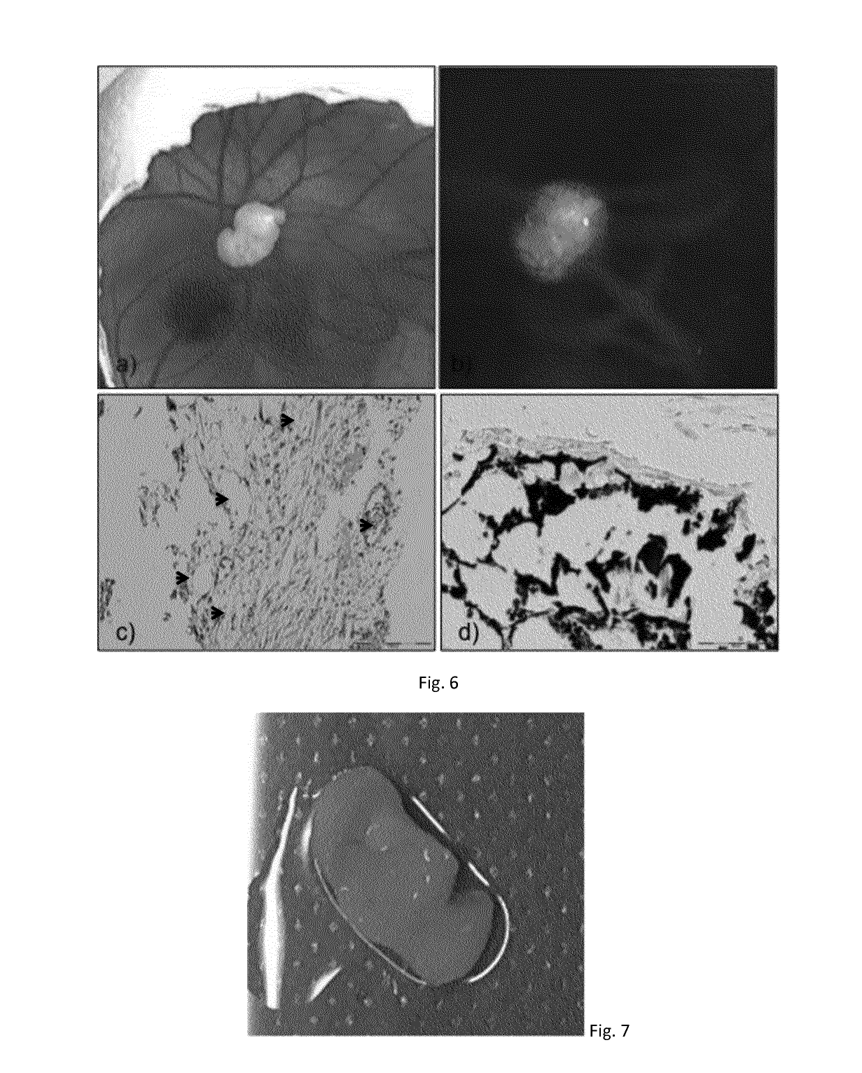

The implant was well integrated and connected to the recipient's vascular system after 5 days in vivo (FIG. 6a,b). Numerous small blood vessels were visible within the graft (FIG. 6c; arrows). Despite the deprivation of differentiation factors, osteogenic differentiation was maintained (FIG. 6d).

### Example 3

### Treatment of Cartilage Lesions

### Graft Harvest and Preparation:

A subcutaneous fat biopsy is harvested under local anaesthesia. This can be done in an outpatient setting approximately 14 days prior to the planned surgical procedure. The fat tissue is aseptically placed in a sterile container containing tissue culture medium and e.g. treated as described above, i.e. the graft is subjected to chondrogenic transdifferentiation for 2 weeks at 37° C., 5% CO₂ and 90% humidity. On the day of the planned procedure, the graft is sent to the operating room. An in vitro transdifferentiated cartilage graft implant is shown in FIG. 7.

### Defect Preparation:

A mini-arthrotomy is performed and the defect is carefully debrided. Using a stencil (e.g. sterile tin foil), an exact mould of the defect is fabricated.

### Graft Preparation and Implantation:

Using the stencil, the graft is fitted to the size of the defect. The graft is then implanted into the defect using fibrin glue. After 5 minutes of hardening time, excessive glue is removed with a scalpel and the joint is flexed and extended completely for 10 times. Stability and position of the graft is inspected during joint movement. Subsequently, the wound is closed.

### Post-Surgical Procedure:

The patients undergo partial weight bearing (10 kg) treatment of the joint for 14 days, afterwards progressive weight bearing depending on swelling. The graft is full weight bearing after about 8 weeks.

### Example 4

### Treatment of a Vertebral Bone Fracture

### Graft Harvest and Preparation:

A subcutaneous fat biopsy is harvested under local anaesthesia. This can be done in an outpatient setting approximately 1-2 weeks prior to the planned surgical procedure. The fat is aseptically dissected into slices of 2 mm² length of edge, placed in a sterile container containing tissue culture medium and e.g. treated as described above, i.e. the graft is subjected to osteogenic transdifferentiation for 1-2 weeks at 37° C., 5% CO₂ and 90% humidity. On the day of the planned procedure, the graft is sent to the operating room.

### Surgical Procure:

The patient is placed in a prone position on a radiolucent table. After determining the location of the incision under fluoroscopy, a stab incision is made. The access instrumentation is inserted and moved forward until pedicle contact is reached. After confirmation of proper trajectory, the instrument is advanced into the vertebral body. Access to the vertebral body can be obtained via guide wire or trocar. Vertebral height can be restored performing a balloon Kyphoplasty procedure if desired.

### Graft Preparation and Implantation:

The graft is delivered in a sterile application device. The application device is connected to the access device. Graft and fibrin are injected simultaneously into the vertebral body under fluoroscopic guidance. After having inserted the desired amount of graft in the vertebral body, the access instruments are removed and the wound is closed.

### Post-Surgical Procedure:

Mobilisation can be started o the day of the procedure. Bracing is recommended until the absence of pain, analgesics should be prescribed as adequate. An exercise program focusing on lumbar stabilisation should be started as soon as permitted by the pain situation.

### Example 5

### Initiation of Neurogenic Transdifferentiation

### Proof of Concept:

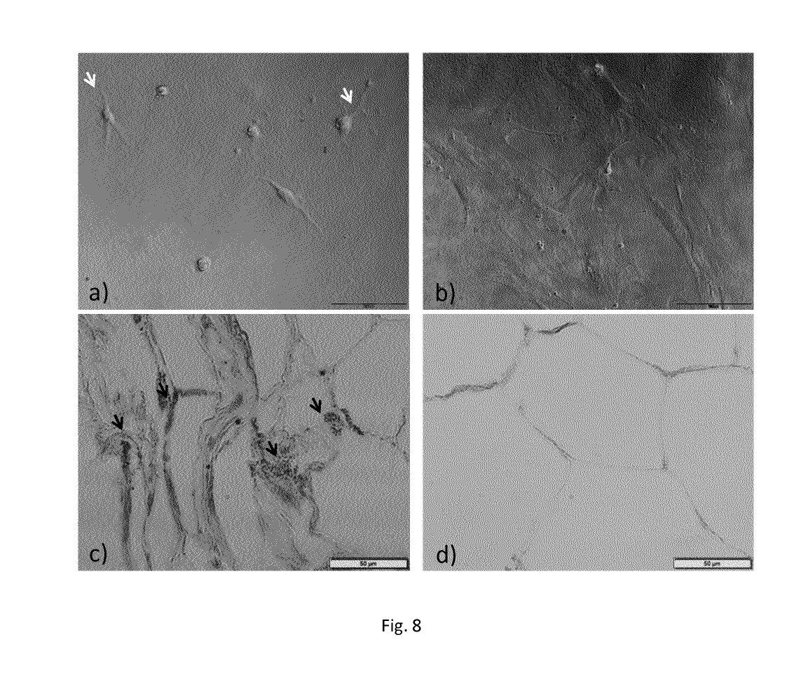

Mesenchymal stem cells were isolated by collagenase digestion from a fat tissue biopsy. Cells were expanded in monolayer culture. After a sufficient amount cells was obtained, cells were plated at a density of 3×10⁴ cells into two wells of a 48 well plate. Neurogenic differentiation was initiated in one well by addition of a commercially available neurogenic differentiation medium. The remaining cells were cultivated in control medium consisting of a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 90% humidity. Medium was exchanged twice a week. After 3 days, a formation of dendrites and axons typical for neuron-like cells was observed (FIG. 8a). Cells cultured in control medium maintained their polygonal shape typical for mesenchymal stem cells (FIG. 8b).

### Fat Graft Preparation:

A small fat biopsy was placed in a sterile container for transportation to the tissue culture laboratory. The sample was washed in sterile saline solution to remove contaminating erythrocytes. After passing the contamination check, the sample was divided into two parts. Part A (Transdifferentiation sample) was incubated in a commercially available neurogenic differentiation medium (Promocell, Heidelberg/Germany) intended to use for mesenchymal stem cell differentiation. Part B (control) was incubated in a 1:1 mixture of Dulbecco's modified Eagle's Medium and Ham's nutrient mix F12 supplemented with 10% Fetal Calf Serum and 2 mM L-glutamine. To prevent bacterial contamination, 50 pg/ml Gentamycin was added to both culture media. Incubation took place at 37° C., 5% CO₂ and 90% humidity for 6 weeks. Medium was exchanged twice a week.

### Histological Evaluation:

At the end of the incubation period, samples were fixed in 4% formaldehyde, washed in phosphate buffered saline and drained in ethanol in ascending concentrations. Tissues were embedded in paraplast and 5 μm sections were prepared. Neurogenic differentiation was evaluated via histochemical stain of Nissl Bodies using Cresyl violet.

### Result:

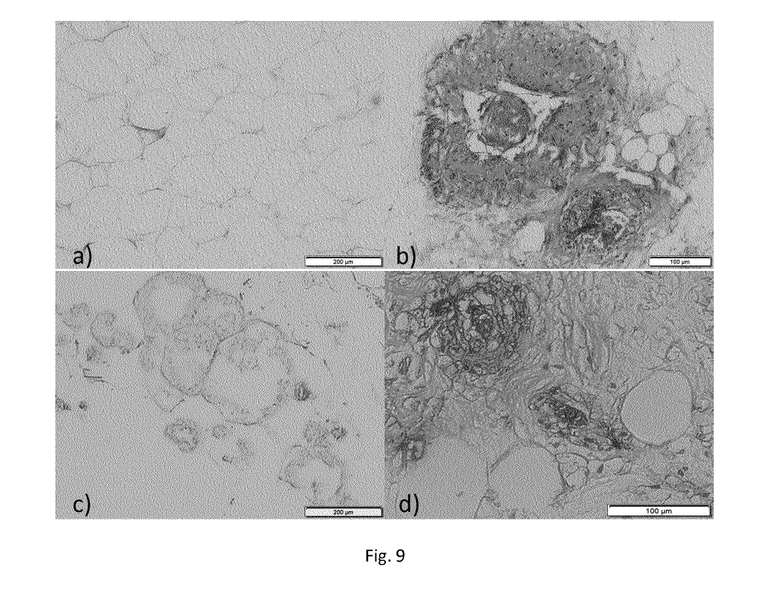

No morphological changes were observed in the control tissue (FIG. 9a). The neurogenic transdifferentiated graft demonstrated positive Cresyl violet staining, indicated by the presence of black-violet Nissl bodies within thy cytoplasm (FIG. 8c, arrows). In the neurogenic transdifferentiated sample, the fat vacuoles were gradually replaced by neurogenic tissue containing round cells with large pericaryons demonstrating positive Cresyl violet staining typical for neural cells (FIG. 9b). The zonal differentiation of peripheral nervous tissue, containing a clearly distinguishable peri- and epineurium surrounding the neural cells as well as concomitant blood vessels, could be observed in the transdifferentiated samples (FIG. 9c). The formation of Nissl bodies was not observed in the control sample (FIG. 8d).

### Example 6

### Induction of Tenogenic Differentiation

### Initial Proof of Concept:

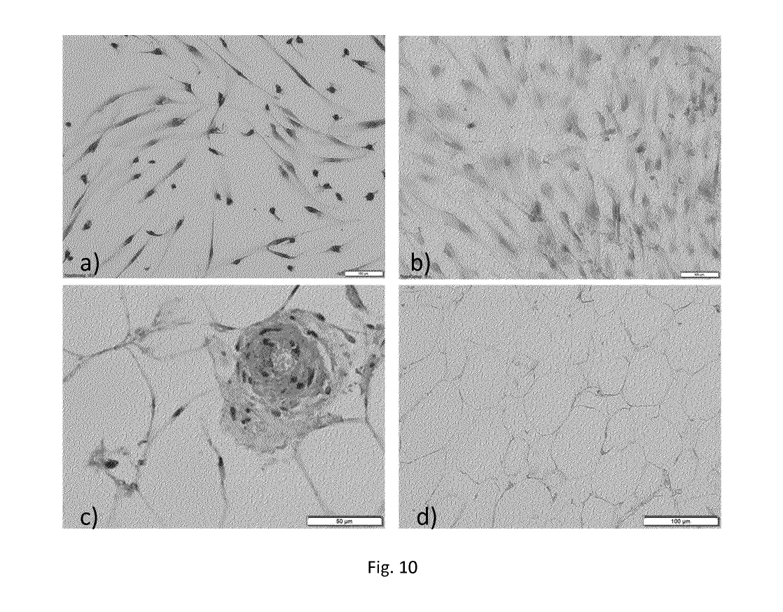

As an initial evaluation of the tenogenic differentiation medium, mesenchymal stem cells were isolated by collagenase digestion from a fat tissue biopsy. Cells were expanded in monolayer culture. After a sufficient amount cells was obtained, cells were plated at a density of 5×10⁴ cells into two wells of a 48 well plate. Tenogenic differentiation was initiated in one well by addition of a tenogenic differentiation medium consisting of DMEM-F12 supplemented with 1% FCS and 10 ng/ml BMP-12. The remaining cells were cultivated in control medium consisting of a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 90% humidity. Medium was exchanged twice a week. Differentiation towards spindle shaped tenocytes was visible in the differentiation group after two weeks (FIG. 10a). No morphological changes were observed in the control group (FIG. 10b).

### Fat Graft Preparation:

A small fat biopsy was placed in a sterile container for transportation to the tissue culture laboratory. The sample was washed in sterile saline solution to remove contaminating erythrocytes. After passing the contamination check, the sample was divided into two parts. Part A (Transdifferentiation sample) was incubated in a tenogenic differentiation medium. Part B (control) was incubated in a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 90% humidity for 6 weeks. Medium was exchanged twice a week.

### Histological Evaluation:

At the end of the incubation period, samples were fixed in 4% formaldehyde, washed in phosphate buffered saline and drained in ethanol in ascending concentrations. Tissues were embedded in paraplast and 5 μm sections were prepared. Tenogenic differentiation was evaluated using H/E staining.

### Result:

Islands of tenocytic differentiated tissue showing circular orientation were present in the transdifferentiated fat pads (FIG. 10c), but not in the control group (d).

### Example 7

### Induction of Myogenic Differentiation

### Initial Proof of Concept:

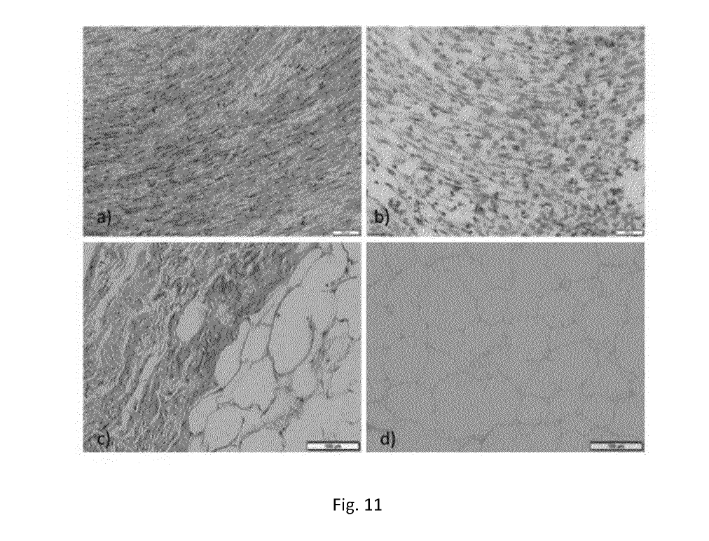

As an initial proof of concept, mesenchymal stem cells were isolated by collagenase digestion from a fat tissue biopsy. Cells were expanded in monolayer culture. After a sufficient amount cells was obtained, cells were plated at a density of 5×10⁴ cells into two wells of a 48 well plate. Myogenic differentiation was initiated in one well by addition of a commercially available myogenic differentiation medium. The remaining cells were cultivated in control medium consisting of a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 90% humidity. Medium was exchanged twice a week. Differentiation towards oriented myocytes were visible after two weeks in the differentiation group (FIG. 11a), but not in the control group (FIG. 11b).

### Fat Graft Preparation:

A small fat biopsy was placed in a sterile container for transportation to the tissue culture laboratory. The sample was washed in sterile saline solution to remove contaminating erythrocytes. After passing the contamination check, the sample was divided into two parts. Part A (Transdifferentiation sample) was incubated in a myogenic differentiation medium. Part B (control) was incubated in a 1:1 mixture of Dulbecco's modified Eagle's Medium and Ham's nutrient mix F12 supplemented with 10% Fetal Calf Serum and 2 mM L-glutamine. To prevent bacterial contamination, 50 μg/ml Gentamycin was added to both culture media. Incubation took place at 37° C., 5% CO₂ and 90% humidity for 6 weeks. Medium was exchanged twice a week.

### Histological Evaluation:

At the end of the incubation period, samples were fixed in 4% formaldehyde, washed in phosphate buffered saline and drained in ethanol in ascending concentrations. Tissues were embedded in paraplast and 5 μm sections were prepared. Myogenic differentiation was evaluated using Masson Goldner staining.

### Result:

After 6 weeks of differentiation, fat vacuoles were partially replaced by muscle tissue demonstrating longitudinal orientation and positive Goldner staining (FIG. 11c). Muscle tissue formation was absent in the control sample (FIG. 11d).

### REFERENCES

¹ Ekanayake E., et al. Acta Neurochir. 152:651-653
² Chien-Lung C. et al. J Chin Med Assoc 69(12):581-584
³ Eder C. et al. Eur Spine J. 2013 May 1. [Epub ahead of print]
⁴ Amini A. R. et al. Crit Rev Biomed Eng. 2012; 40(5): 363-408.
⁵ Yang Y. et al. Med Sci Monit. 2013; 19: 826-836
⁶ Robinson Y. Patient Safety in Surgery 2008, 2:2
⁷ Thomsaon L A. Biomaterials 1992; 13(12):811-8.
⁸ Schmelzer-Schmied N. Eur Spine J 2009; 18:624-629
⁹ Blattert T R. Spine (Phila PA 1976) 2009 Jan. 15; 34(2):108-14
¹⁰ Pridie K H. J Bone Joint Surg Br. 1959; 41: 618.
¹¹ Steadman J R, et al. Clin Orthop Relat Res. 2001; 391 Suppl: 362-369
¹² Gomoll A H, et al. Knee Surg Sports Traumatol Arthrosc. 2010: 18(4):434-447
¹³ Hangody L., et al. Orthopedics. 1998; 2(7):751-756
¹⁴ Orth P. et al. Stem cells and Cloning: Advances and Applications 2014, 7:1-17
¹⁵ Brittberg M., et al. N Engl J Med. 1994; 331(14):889-895
¹⁶ National Institute for health and clinical excellence: The use auf autologous chondrocyte implantation for the treatment of cartilage defects in knee joints. Review of Technology Appraisal 16, May 2008 www.nice.org.uk/nicemedia/pdf/TA089guidance.pdf
¹⁷ Niemeyer P. et al. Am J Sports Med. 2008 November; 36(11):2091-9
¹⁸ Pietschmann M. F. et al. Am J Sports Med. 2012 January; 40(1):68-742
¹⁹ Safety information Carticel®, www.carticel.com
²⁰ Solchaga L. A., et al. Methods Mol. Biol. 2011, 698:253-278
²¹ Jaiswal N et al. J Cell Biochem. 1997 February; 64(2):295-312.
²² Halvorsen Y C et al. 2000; 24, Suppl 4:S41-S44
²³ Evans et al., European Cells and Materials 2009; 18:96-111
²⁴ Evans, Injury 2011 June; 42(6): 599-604 as NIH Public Access manuscript of 1 Jun. 2012
²⁵ Orlicky D. J., Schaack J. J Lipid Res 2001; 42:460-466
²⁶ Wang et al., Genet. Mol. Res. 13 (2) (2014): 4599-4606
²⁷ Sandor et al., Journal of oral and maxillofacial surgery 71 (5) (2013): 938-950
²⁸ Salibian et al., Archives of plastic surgery 40 (6) (2013): 666
²⁹ Inok Kim et al., Tissue engineering part a 19 (21-22) (2013): 2372-2381
³⁰ Jung et al., The journal of craniofacial surgery 21 (2) (2010): 468-462
³¹ Eun Hee et al., World journal of stem cells 6 (1) (2014): 65
³² Stromps et al., Biomed research international 111 (1) (2014): 79-7
³³ Sujeong et al., BMC cell biology 11 (1) (2010): 25

1-17. (canceled)

18. A method of producing a bioengineered connective tissue graft by direct transdifferentiation of a donor connective tissue, preferably fat tissue, into another connective tissue type comprising contacting the donor connective tissue in vitro or in vivo by one or more administered connective tissue specific growth or differentiation factor.

19. A method of producing a connective tissue graft suitable for correcting a connective tissue defect, comprising deter-mining the size and shape of a tissue defect, treating a donor connective tissue, preferably fat tissue, obtained from a patient by, in any order: modelling donor connective tissue, preferably fat tissue, to fit the size and shape of the tissue defect and contacting the donor connective tissue, preferably fat tissue, with one or more connective tissue specific growth or differentiation factors, thereby initiating differentiation of the tissue graft into another connective tissue.

20. The method of claim 18 further comprising placing the differentiated tissue into the tissue defect of a patient, preferably wherein the patient with the tissue defect and the patient providing the donor tissue is the same patient (autologous tissue).

21. The method of claim 18 wherein the step of culturing the donor connective tissue or the contacting step is for at least 1 hour, at least 1 day, preferably at least 2 or at least 3 days, especially preferred at least 4 days.

22. The method of claim 18, wherein the another connective tissue is bone, cartilage, muscle, tendon, ligament or nerve, preferably bone or cartilage.

23. The method of claim 18, wherein the connective tissue is cartilage and differentiation comprises the differentiation into chondrocytes and/or chondroblasts, with the differentiation factor being a chondrocyte differentiation factor, preferably wherein said factor includes TGF-beta.

24. The method of claim 18, wherein the connective tissue is bone and differentiation comprises the differentiation into osteocytes and/or osteoblasts, with the differentiation factor being an osteogenic differentiation factor, preferably wherein said factor includes beta-glycerophosphate.

25. The method of claim 18, wherein connective tissue specific growth or differentiation factors comprise ascorbic acid or an ascorbic acid ester, preferably ascorbate-2-phosphate, or any pharmaceutically acceptable salt thereof.

26. The method of claim 18, wherein the fat tissue comprises stromal cells and adipocytes, such as white and/or brown adipocytes.

27. The method of claim 18, wherein the differentiated tissue graft is inserted into a tissue defect and/or wherein the differentiated tissue graft is inserted into an intervertebral disc compartment or into a cage designed for insertion into the intervertebral disc compartment for spinal fusion, and further preferably wherein the insertion is fixed by a tissue sealant, preferably a fibrin glue.

28. The method of claim 18 further defined as an ex vivo method for preparing a donor, preferably fat, tissue into a differentiated graft suitable for connective tissue repair, comprising contacting the donor, preferably fat, tissue with one or more connective tissue specific growth or differentiation factors, thereby initiating differentiation of the tissue graft, wherein said contacting is for a time period of between 1 hour to 6 weeks at a temperature of between 30 to 40° C., 0.01% to 10% (w/v) CO₂ and between 70% to 98% humidity, wherein the connective tissue specific growth or differentiation factors is not a nucleic acid or consist of proteins, peptides and small molecules with a size of at most 10 kDa.

29. The method of claim 18 further comprising contacting the tissue with a tissue sealant, preferably after treatment with said connective tissue specific growth or differentiation factors.

30. A connective tissue specific growth or differentiation factor for use in a method of claim 18.

31. A kit suitable for performing a method of claim 27 comprising a connective tissue specific growth or differentiation factor and a tissue sealant, preferably a fibrin glue.

32. The kit of claim 31 further comprising a cartilage or bone tissue label or marker.

33. Use of the kit of claim 31 comprising the insertion is fixed by the tissue sealant.
